(19) United States
(12) Patent Application Publication
(10) Pub. No.: US 2021/0100935 A1
(43) Pub. Date: Apr. 8, 2021

(54) ENGINEERED HEPARIN BIOACTIVE MATRIX FOR CLINICAL APPLICATION OF BLOOD CONTACTING SURFACE AND METHOD OF MANUFACTURING THE SAME

(71) Applicant: Ension, Inc., Butler, PA (US)

(72) Inventors: Ali Hussain, Cape Coral, FL (US); Linda Cahalan, Cape Coral, FL (US); Patrick Cahalan, Cape Coral, FL (US)

(21) Appl. No.: 17/074,888

(22) Filed: Oct. 20, 2020

Related U.S. Application Data
(63) Continuation of application No. PCT/US2018/061993, filed on Nov. 20, 2018.
(60) Provisional application No. 62/660,804, filed on Apr. 20, 2018.

(51) Int. Cl. A61L 33/08; A61L 33/00

(52) U.S. Cl. CPC A61L 33/08; C08L 5/10; A61L 2400/18; A61L 33/0017

(57) ABSTRACT

A method of manufacturing a medical product having an engineered heparin bioactive matrix for clinical application on a blood contacting surface comprises: a) activating a blood contacting surface of at least one component of a medical device via one of plasma treatment or gas activation; b) assembling the medical product; c) Setting up medical device for wet chemistry in which wet chemistry treatments follows a blood flow path through device; d) enhancing at least the blood contacting surface with a wet chemistry treatment including an aqueous solution having a strong oxidizing agent, such as ammonium persulfate; e) adding a positively charged spacer molecule to at least the blood contacting surface with a wet chemistry treatment including an aqueous solution having a cationic polymer, such as PEI; and f) covalently immobilizing heparin to at least the blood contacting surface with a wet chemistry treatment including heparin, preferably deaminated heparin.

### RELATED APPLICATIONS

The present application claims the benefit of international patent application serial number PCT/US2018/061993 filed Nov. 20, 2018 and published Oct. 24, 2019 as WO 2019/203898, which application and publication are incorporated herein by reference. International patent application serial number PCT/US2018/061993 claims the benefit of U.S. Provisional Application Ser. No. 62/660,804 entitled “Engineered Heparin Bioactive Matrix for Clinical Application of Blood Contacting Surface and Method of Manufacturing the Same” which is incorporated herein by reference.

### BACKGROUND OF THE INVENTION

### Field of the invention

The present invention relates to surface treated blood contacting medical devices such as blood oxygenators.

### Background Information

Devices used in the medical field must be manufactured using materials having biocompatibility, also called biomaterials, having particular surface properties so that the device functions without causing adverse effects to the patient. Technically, according to Black's Medical Dictionary, 2010, biocompatibility is defined as the ability of a biomaterial to perform its desired function with respect to a medical therapy, without eliciting any undesirable or systemic effects in the recipient or beneficiary of that therapy, but generating the most appropriate beneficial cellular or tissue response in that specific situation, and optimizing the clinically relevant performance of the therapy. The definition is somewhat looser in practice in that the strict application of the definition of “generating the most appropriate beneficial cellular or tissue response” is not always achieved with materials that are classified as biomaterials and sometimes a material is deemed biocompatible if there is no immediate catastrophic adverse tissue response or immediately observable increased patient mortality.

Biomaterials are typically made of inert metals, polymers, or ceramics to ensure durability and to try to have the materials not adversely react with the physiological environment with which they come into contact, such as with blood or tissues. More particularly, many biomedical devices may or may not require blood compatible, infection resistant, and/or tissue compatible surfaces. For example, it is often desirable to manufacture medical devices, such as catheters, that have properties that discourage adherence of blood or tissue elements to the device. Adverse reactions between materials and blood components are predominant factors limiting the use of synthetic materials that come into contact with physiological fluids.

A number of approaches have been suggested to improve the biocompatibility and blood compatibility of medical devices. One approach has been to modify the surface of the material to prevent undesirable protein adhesion by providing the material with a low polarity surface, a negatively charged surface, or a surface coated with biological materials, such as enzymes, endothelial cells, and proteins. Another approach has been to bind anticoagulants to the surface of biologically inert materials to impart anti- or non-thrombogenic characteristics to the materials. Still another approach used in the art has been the copolymerization of various phospholipids which are used as coating materials for various substrates. Partial polymeric backbone coatings have also been used in a similar fashion. However, many of these methods can result in a leaching or “stripping off” of the coating.

In devices requiring the transfer of gases, for example, in blood oxygenators requiring the exchange of oxygen and carbon dioxide through a membrane or porous fiber, there are additional drawbacks. Often surfaces that have been rendered biocompatible by the coating of biomolecules attract phospholipids. Phospholipids that adhere to the surface coat the pores and wet the surface of the device, making it hydrophilic. Water adversely affects gas transfer, making the oxygenator significantly less effective. There remains a need in the art to develop processes for preparing substrates coated biomolecules that demonstrate biocompatibility and blood compatibility, while maintaining gas permeability.

The patent literature details general oxygenator operation and construction. U.S. Patent Application Publication Numbers 2017-0312416, 2017-0007755, 2016-0136347, 2015-0182681, 2014-0037500, 2013-0343954, 2013-0296633, 2013-0094997, 2010-0272602, 2008-0199357, 2007-0249888, 2005-0042141 and 2002-0057989 are representative examples which are incorporated herein by reference. Many of the above systems relate to an integrated centrifugal blood pump-oxygenator having a rotational body being rotatable in a rotor-housing, often located on the bottom.

Lung diseases are the third largest cause of death in the United States of America, accounting for approximately 1 out of every 7 adult deaths. It has been estimated that 30 million Americans are living with chronic lung disease. Adult respiratory distress syndrome (ARDS) has been reported to afflict approximately 150,000 patients annually in the U.S, and despite advances in critical care, mortality remains around 40-50%. Currently available therapies for patients with chronic respiratory failure include, for example, ventilation and extracorporeal membrane oxygenation (ECMO). In intensive care medicine, extracorporeal membrane oxygenation (ECMO) is an extracorporeal technique of providing both cardiac and respiratory support oxygen to patients whose heart and lungs are so severely diseased or damaged that they can no longer serve their function.

ECMO systems attempt to closely simulate physiological gas exchange. Attempts have been made to integrate multiple components of cardiopulmonary, ECMO-systems into single structures so as to eliminate or minimize the need for the extension of lengthy, blood-filled tubes. Various integrated pump-oxygenators have been described as discussed in U.S. Pat. Nos. 5,217,689; 5,266,265; 5,270,005; 5,770,149; 4,975,247; 5,395,468; 5,429,486; 6,963,222; and 6,730,267. U.S. Pat. No. 4,639,353 discloses the use of an arrangement of bundles of hollow fibers perpendicular to the direction of blood flow via a series of flow guide structures. U.S. Pat. No. 5,263,924 discloses the use of an integrated centrifugal pump and membrane oxygenator comprising hollow fibers, which are displaced circumferentially in a ring around an impeller of the centrifugal pump and through which blood is pumped for oxygenation. See also U.S. Pat. Nos. 3,998,593 and 4,490,331 which are incorporated herein by reference. Other efforts to decrease the effect of the boundary layer include actively rotating hollow fiber membranes or moving fiber membranes in the path of blood flow, examples of which are described in U.S. Pat. Nos. 5,830,370; 6,217,826; 6,503,450; 6,723,284; and 7,122,155. Additional patents and publications giving a general overview of conventional blood pump-oxygenators include U.S. Pat. Nos. 4,239,729, 6,929,777, and 7,927,544, and U.S. Publication Nos. 2004-0219059, 2008-0190870, 2009-0175762, 2010-0101657, 2010-0288703, 2016-0296685 and 2017-0021081 which are incorporated herein by reference.

U.S. Publication 2015/0352265, which is incorporated herein by reference, discloses hemo-compatible, anti- and non-thrombogenic, heparin-based bioactive coatings for a surface to be contacted with blood, such as a surface of an oxygenator device (also called a Hollow Fiber Membrane (HFM) surface) or an artificial lung, which coatings include a quaternary ammonium salt and heparin complex (QUAT). According to representative embodiments, the surface is treated with polyvinylpyrolidone (PVP), followed by a coating layer of said quaternary ammonium salts and heparin complex (QUAT) to form a PVP-QUAT coating. According to an alternative embodiment, anionic functional groups are created on the one or more surfaces of the HFM by modifying the surface of the HFM using ionic complexes dissolved in a solvent mixture that includes major quantity of alcohol along with a minor quantity of organic dissolving agents. None of the approaches presented yield optimized Physiologically Significant Bioactivity and Stability.

A surface treatment approach for treating medical devices is described in U.S. Pat. No. 8,114,465 and U.S. Patent Application Publication Numbers 2008-0241349 in which a typical process comprises: a) providing a substrate, such as fibers for an oxygenator; b) coating the substrate with a polysiloxane or acrylomide; c) rendering the polysiloxane and/or acrylamide surface amino functional; and d) contacting the amino-functional polysiloxane surface with a biomolecule, such as heparin under conditions effective to attach the biomolecule to the substrate. This surface modification or treatment approach has distinct advantages over typical coating approaches, but can be further optimized for increased stability and bioactivity and scalability.

The remains a need for an engineered bioactive heparin matrix with optimized physiologically significant bioactivity and increased stability suitable for clinical applications and universally applicable methods of manufacturing the same

### SUMMARY OF THE INVENTION

One aspect of this invention is directed to a method of manufacturing a medical product having an engineered heparin bioactive matrix for clinical application on a blood contacting surface comprising the steps of: a) activating a blood contacting surface of at least one component of a medical device via one of plasma treatment or gas activation; b) assembling the medical product; c) Setting up medical device for wet chemistry in which wet chemistry treatments follows a blood flow path through device; d) enhancing at least the blood contacting surface with a wet chemistry treatment including an aqueous solution having a strong oxidizing agent, such as ammonium persulfate; e) adding a positively charged spacer molecule to at least the blood contacting surface with a wet chemistry treatment including an aqueous solution having a cationic polymer, such as PEI; and f) covalently immobilizing heparin to at least the blood contacting surface with a wet chemistry treatment including heparin, preferably deaminated heparin.

These and other advantages are described in the brief description of the preferred embodiments in which like reference numeral represent like elements throughout.

### BRIEF DESCRIPTION OF THE FIGURES

FIG. 1 is a schematic view of a manufacturing facility for forming a medical device for clinical application having an engineered heparin bioactive matrix of blood contacting surface in accordance with one aspect of the present invention.

### DESCRIPTION OF THE PREFERRED EMBODIMENTS

It is noted that, as used in this specification and the appended claims, the singular forms “a,” “an,” and “the” include plural referents unless expressly and unequivocally limited to one referent.

For the purposes of this specification, unless otherwise indicated, all numbers expressing quantities of ingredients, reaction conditions, and other parameters used in the specification and claims are to be understood as being modified in all instances by the term “about.” Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

All numerical ranges herein include all numerical values and ranges of all numerical values within the recited numerical ranges. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The various embodiments and examples of the present invention as presented herein are each understood to be non-limiting with respect to the scope of the invention.

As used in the following description and claims, the following terms have the indicated meanings:

The terms “on”, “appended to”, “affixed to”, “bonded to”, “adhered to”, or terms of like import means that the designated item, e.g., a coating, film or layer, is either directly connected to (superimposed on) the object surface, or indirectly connected to the object surface, e.g., through one or more other coatings, films or layers (superposed on).

The terms “attach”, “couple”, and “link” refer to securing a material or biomolecule to a substrate, for example, by chemical covalent or ionic bonding, such that the coating or biomolecule is immobilized with respect to the substrate.

The term “medical device” is a device that has at least one surface that contacts tissue, blood, or other bodily fluids in the course of its operation, which fluids are present within or subsequently introduced into patients and which has an internal blood/fluid flow path through the device. This definition can include, for example, extracorporeal devices for use in surgery such as blood oxygenators, blood pumps, blood sensors, tubing used to carry blood and the like which contact blood which is then returned to the patient. This can also include endo-prostheses implanted in blood contact in a human or animal body such as vascular grafts, stents, heart valves, and the like that are implanted in blood vessels or in the heart. This can also include devices for temporary intravascular use such as catheters, dialysis machine, and the like which are placed into the blood vessels or the heart for purposes of monitoring or repair.

The term “biomolecule” refers to a biologically active molecule.

A “biocompatible” material does not generally cause significant adverse reactions (e.g., toxic or antigenic responses) in the body, whether it degrades within the body, remains for extended periods of time, or is excreted whole (see ISO 10993).

Ideally, a biocompatible material will not induce undesirable reactions in the body as a result of contact with bodily fluids or tissue, such as infection, coagulation, tissue death, tumor formation, allergic reaction, foreign body reaction (rejection) or inflammatory reaction.

A “blood compatible” material is one that will not induce undesirable reactions in the body as a result of contact with blood, such as blood clotting or infection. A blood compatible material is understood to be biocompatible.

The present invention also is particularly applicable to blood gas exchange devices, e.g., oxygenators which, in general are extremely well known in the art, as fully described in the above cited patents and published patent applications that are incorporated herein by reference. The present invention is particularly well suited for both well-known sheet and tubular forms of membrane oxygenators. In a membrane oxygenator, the blood is separated from direct contact with the oxygenating gas by a membrane, which is disposed within a hollow housing. This membrane is microporous or semipermeable, that is, capable of permitting carbon dioxide and oxygen to permeate through it while at the same time preventing the blood itself from passing there through.

The tubular oxygenators are also known or referred to as hollow fiber oxygenators, and the general format is illustrated in U.S. Pat. No. 4,239,729. A hollow fiber oxygenator employs a large plurality (typically thousands) of microporous or semipermeable hollow fibers disposed within a housing. These hollow fibers are sealed in the end walls of the housing; the end walls are then fitted with skirted end caps. One end cap is fitted with an inlet, and the other is fitted with an outlet. In the '729 patent oxygenator, the hollow fibers are aligned in the housing so that their longitudinal axes are generally parallel to the longitudinal axis of the housing. In this device, blood enters through the inlet of one end cap, passes through the lumens of the hollow fibers, and exits through the outlet of the other end cap. Oxygenated gas enters the device through the inlet in the peripheral wall near one end of the device, passes over the outer surfaces of the hollow fibers, and exits the device through the outlet in the peripheral wall near the other end of the device. It will be understood that carbon dioxide diffuses from the blood flowing inside the hollow fibers through the fiber walls into the stream of oxygenating gas. At the same time, oxygen from the oxygenating gas flowing over the outer surfaces of the hollow fibers diffuses through the walls of the hollow fibers into the lumens thereof to oxygenate the blood flowing there through. Other oxygenators comprising hollow fibers have been developed. These oxygenators typically comprise a plurality of hollow fibers disposed within a hollow housing and arranged so that blood typically flows over the hollow fibers and gases typically flow through the hollow fibers. Many configurations are possible as to the direction of fluid flow and the arrangement of fibers. The fibers may be in a linear, circular, or spiral arrangement, for example, or may be wrapped or wound around a core in various configurations. Hollow fiber membrane oxygenators are described, for example, in U.S. Pat. Nos. 4,975,247 and 5,395,468, which are incorporated herein by reference.

A second type of membrane oxygenator, called the flat plate membrane oxygenator, employs one or more thin, flat sheets of microporous membrane. In its most basic form, the flat plate oxygenator has a single sheet of microporous membrane sealed into a housing so as to provide in the housing a first compartment (the “blood compartment”) for the flow of blood, and a second compartment (the “gas compartment”) for the flow of an oxygenating gas. Each of the compartments is fitted with an inlet and an outlet. Blood flows into and out of the blood compartment and the oxygenating gas flows into and out of the gas compartment. Oxygen passes from the oxygenating gas across the membrane into the blood flowing through the blood compartment. Carbon dioxide passes from the entering blood across the membrane to be entrained in the oxygenating gas. The exiting blood, now reduced in carbon dioxide and enriched in oxygen, is returned to the patient.

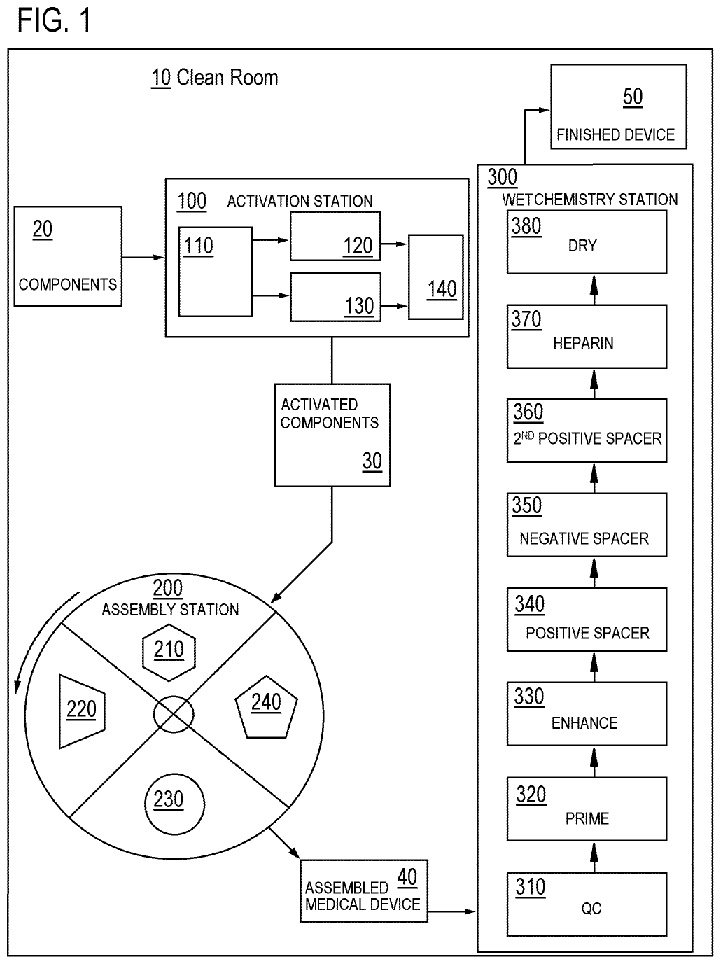

FIG. 1 is a schematic view of a manufacturing facility for forming a medical device 50 for clinical application having an engineered heparin bioactive matrix of blood contacting surface in accordance with the present invention. The medical device 50 is preferably one of a blood oxygenator, a dialysis catheter and a dialysis machine, and more preferably the medical device 50 is a tubular blood oxygenator as generically described above. The present invention is related to an engineered heparin bioactive matrix on a blood contacting surface of the medical device 50 as described below, but the present method and manufacturing system may be used for other biomolecules. An engineered heparin bioactive matrix, or any engineered bioactive matrix within the meaning of this application refers to a substrate which has undergone surface treatment in accordance with engineering principles of maintaining homeostasis (See ISO 10993-4), and specific to blood contacting surfaces, maintaining hemostasis, namely maintaining the ability of the blood to maintain normal function as determined or verified by measuring the molecular and cellular elements of the blood maintaining within accepted clinical parameters. The engineered heparin bioactive matrix of blood contacting surface in accordance with present invention yields clinically relevant Physiological biofunctionality (bioactivity) which is stable over a long period of time.

In summary, the present invention provides a method of manufacturing a medical product 50 for clinical application having an engineered heparin bioactive matrix on a blood contacting surface in which components 20 have surface activation of select surfaces at an activation station 100; the activated components 30 of the device 50 are forwarded to an assembly station 200, and the assembled medical device 40 is forwarded to a wet chemistry station 300 for application of having an engineered heparin bioactive matrix on a blood contacting surface to form the finished medical device 50. The essential steps include a) activating a blood contacting surface of at least one component of a medical device 50 via one of plasma treatment 120 or gas activation 130; b) assembling the medical product at station 200; c) setting up the assembled medical device for wet chemistry (310, 320) in which wet chemistry treatments follows a blood or fluid flow path through device 50; d) enhancing at step 330 at least the blood contacting surface with a wet chemistry treatment including an aqueous solution having a strong oxidizing agent; e) adding a positively charged spacer molecule at step 340 to at least the blood contacting surface with a wet chemistry treatment including an aqueous solution having a cationic polymer; and f) covalently immobilizing heparin at step 370 to at least the blood contacting surface with a wet chemistry treatment including heparin.

The manufacturing process of the present invention is configured for being performed within a single clean room 10. Cleanroom 10 is an enclosed and environmentally-controlled space in which temperature, humidity, pressure and contaminant levels are kept within defined limits. The controlled environment provided by the clean room 10 helps to ensure that products 50 remain under controlled contamination levels throughout the production process, thereby reducing potential risks to patients. The single clean room 10 for the process of the present invention is beneficial for the production of safe devices 50. Details for the construction and operation of the clean room 10 are set forth in ISO 14644.

The components 20 forming the medical device 50 are brought into the clean room 10 and to an inspection station 110 at the activation station 100. Following the inspection the individual components 20 typically goes onto the activation via plasma treatment 120 or gas activation 130, or may go to a cleaning step if the inspection identifies an issue, or the component is discarded if a cleaning cannot remedy the problem with the component 20. Any component 20 of the device 50 must pass its own inspection according to the associated product 50 protocol. It is possible that some components 20 will undergo a pre-Cleaning before the clean room to assure that the component 20 is beginning from a desired state, and such pre-cleaning may be useful if there is variability from manufacturing or multiple vendors.

The components 20 passing inspection at step 110, which may be cleaned and inspected again, move onto activating a blood contacting surface of at least one component 20 of the medical device 50 via one of plasma treatment 120 or gas activation 130.

Plasma treatment 120 take place within a plasma chamber containing electrodes, across which a voltage is applied. The chamber may be partially evacuated. The plasma treatment of the present invention may be with propene, oxygen, siloxane (hexamethyldisiloxane-HMDSO and/or tetramethyldisiloxane-TMDSO) or acrylic acid, in which a stream of the relevant gas is fed into the chamber. When a high frequency voltage is applied between the electrodes, current flows into the chamber, forming a plasma, which is a glowing electrical discharge within the gas. Reactive chemical species are formed in this electrical discharge. Plasma treatment 120 has certain advantages over gas treatment 130 and may be preferable for polycarbonate substrates.

Gas activation 130 within the meaning of this application is ozone activation or ozone treatment also known as UV ozone treatment. Gas activation 130 may be done at ambient pressures. Gas activation 130 may be preferable for polypropylene (PP), polyurethane (PU) polyethylene (PE) and silicone substrates.

Following surface activation via one of plasma treatment 120 or gas activation 130, the component is moved to performance testing 140 in which representative components 20 are inspected and tested using conventional testing such as one of FTIR, SEM Contact angle and XPS. Following passing of the performance testing 140 the activated components 30 are forwarded to the assembly station 200. The record of the performance testing 140 is maintained to establish a quality review record of the production of the finished device 50, or for a batch of the finished devices 50.

The activated components 30 are assembled into the medical product 40 at station 200, specifically at assembly station 240. The present invention is particularly well suited to application where the medical device 50 is one of a blood oxygenator, a dialysis catheter and a dialysis machine. Regarding blood oxygenators the present invention is well suited for forming tubular blood oxygenators. In the formation of a tubular blood oxygenator as the medical device 50, the first step of the assembly station 200 is a winding apparatus or station 210 forming a tubular fiber bundle for the tubular blood oxygenator. The winding station may also be referred to as fiber bundling which is the winding of the hollow (often polypropylene although Polymethalpentene PMP is commonly used as oxygenation membranes) fibers into the fiber bundle that forms the central core of a membrane oxygenator. The details of the winding device and the formed bundle will depend on the particulars of the oxygenator housing and operational parameters and desired surface area and other specifications, however for some general background see “Fiber manufacturing, membrane classification, and winding technologies associated with membrane oxygenators” by Schaadt J. J Extra Corpor Technol. 1998 Mar; 30(1):0-4.

Following the winding station 210 is a potting station 220 for the tubular fiber bundle in which the tubular fiber bundle is placed in a centrifugal potting apparatus for proper distribution of the potting material (adhesive or resin). Following the potting station 210 is a toming station or trimming station in which the potted fiber bundle is finished for assembly. The finished fiber bundle is moved, with the remaining components of the tubular blood oxygenator to the assembly station 240 for assembling of the trimmed potted fiber bundle and other components of the tubular blood oxygenator. Components are assembled at the station 240 to form the assembled medical device which can be moved to the wet chemistry station 300. The assembly station 200 may be efficiently formed in a compact arrangement as an index able turntable, or a stationary platform with a central pick and place robot. The process of the invention remains substantially unchanged if workers manually advance the components along through the station 200. The assembly station 200 will be constructed significantly different for different medical products, as non-tubular oxygenators, or catheters will not have the winding 210, potting 220 or toming 230 stations as will be evident. Other sub-assembly steps can be incorporated into the station 200 as appropriate for the specific medical device 50.

The assembled medical device 40 with some activated surfaces is moved to the wet chemistry station 300 for wet chemistry treatments which will follow a blood or fluid flow path through device 50 The first step is setting up the assembled device 40 for wet chemistry with in-line quality control at step 310. The present invention contemplates a batch process for manufacturing multiple devices 50 simultaneously. The devices 40 must be set up such that all devices 40 receive uniform treatment to assure uniform finished devices 50. In this context parallel set ups rather than serial alignment of devices 40 is preferred, as serial alignment of devices 40 can yield lower treatment yields/levels for downstream devices and unacceptable lack of uniformity between devices. The set up includes coupling the devices to source of wet chemistry treatments. Additionally the present invention provides for in-line quality control through the visual staining of sacrificial inline tubing section, as desired. In other words the present method includes additional inline sacrificial tubing for processing device 50 (or set of devices 50). The tubing will be selectively removed and stained to determine treatment effectiveness and provide a verifiable record of quality control. The initial sacrificial tubing section of step 310 represents the baseline before wet chemistry treatments have started on the connected device 40 for creating a quality review record of the process for the device 50 (as the quality review is determined to be needed).

The next step 320 is a water priming step which is also used for checking for leaks or undesired blockage in the set up to verify the set up arrangement. The water priming is accomplished at ambient or room temperature and is also utilized for calculating priming volume for the coupled devices 40 and treatment apparatus.

Following the priming step of 320 is the enhancing step 330 for enhancing at least the blood contacting surface with a wet chemistry treatment including an aqueous solution having a strong oxidizing agent, preferably Ammonium persulfate (NH₄)₂S₂0₈. Specifically, [1-15%] (NH₄)₂S₂0₈ in (deionized) DI H₂O. The ammonium persulfate will enhance the activated and non-activated component surfaces of the medical device 40 as Ammonium persulfate assists in activation of PVC and polycarbonate which may represent other components of a tubular oxygenator other than the fiber bundle that is treated as described above. The Enhancing wet chemistry treatment of step 330 is preferably at an elevated temperature of at least 50° C., preferably 60° C.-65° C., and is recirculated through the device(s) 40 for a circulation time of 5-45 minutes, preferably 15-30 minutes. The enhancing step 330 then includes a non-recirculating rinse of about 3× priming volume of DI water at room temperature.

Following the enhancing step 330 is a step 340 of coupling a positive spacer, namely adding a positively charged spacer molecule to at least the blood contacting surface with a wet chemistry treatment including an aqueous solution having a cationic polymer. Preferably this cationic polymer solution is a 0.001 to 0.1% w aqueous solution of positively charged spacer polyethylenimine (PEI) molecule with cross-linker, preferably crotonaldehyde (CH₃CH═CHCHO) in buffer at a pH of about PH9. The cross linker crotonaldehyde may be present in concentrations of about about 100-500μ/liter solution. The positive spacer wet chemistry treatment of step 340 is preferably ambient or room temperature and is recirculated through the device(s) 40 for a circulation time of 5-45 minutes, preferably 15-30 minutes. The positive spacer wet chemistry treatment of step 340 then includes a non-recirculating rinse of about 3× priming volume of DI water at room temperature.

The positive spacer wet chemistry treatment of step 340 then may include a quality control check of taking an in-line sacrificial tube section and stain the section and maintaining the results as a record of the wet chemistry process. This section of step 340 can be objectively compared to the baseline section taken in step 310. The quality review steps discussed here can be accomplished with every process for every device 50, but in typical practice it may be accomplished on selected devices 50 (often a given total number of quality reviews, say 10-30, at random intervals for every 1000 units produced). The main advantage of the quality review of the present invention is that it provides objective analysis without sacrificing production units of devices 50. The final medical devices 50 formed in manufacturing may have a certain number sacrificed for other regulatory requirements which this quality control paradigm might not satisfy and the present quality review paradigm allows the present methodology to go beyond the minimum reviews required without sacrificing additional units 50.

Following the positive spacer wet chemistry treatment of step 340 is a step 350 of coupling a positive spacer, namely adding a negatively charged spacer molecule to at least the blood contacting surface with a wet chemistry treatment including an aqueous solution having an anionic polymer. Preferably this anionic polymer solution is a, namely 0.001 to 0.1% w of negatively charged spacer such as Dextran sulfate in saline solution pH 3. The negative spacer wet chemistry treatment of step 350 is preferably at an elevated temperature of at least 50° C., preferably 60° C.-65° C., and is recirculated through the device(s) 40 for a circulation time of 5-45 minutes, preferably 15-30 minutes. The negative spacer wet chemistry treatment of step 350 then includes a non-recirculating rinse of about 3× priming volume of DI water at room temperature. The negative spacer wet chemistry treatment of step 350 then may, if desired, include a quality control check of taking an in-line sacrificial tube section and stain the section and maintaining the results as a record of the wet chemistry process.

Following the negative spacer wet chemistry treatment of step 350 is a step 360 of coupling a 2^{nd} positive spacer, namely adding a positively charged spacer molecule to at least the blood contacting surface with a wet chemistry treatment including an aqueous solution having a cationic polymer. Preferably this cationic polymer solution is a 0.001 to 0.1% w aqueous solution of positively charged spacer (PEI) molecule (with no cross-linker) in DI water at a pH of about PH9. The 2^{nd} positive spacer wet chemistry treatment of step 360 is preferably ambient or room temperature and is recirculated through the device(s) 40 for a circulation time of 5-45 minutes, preferably 15-30 minutes. The 2^{nd} positive spacer wet chemistry treatment of step 360 then includes a non-recirculating rinse of about 3× priming volume of DI water at room temperature. The 2^{nd} positive spacer wet chemistry treatment of step 360 then may include a quality control check, if desired, of taking an in-line sacrificial tube section and stain the section and maintaining the results as a record of the wet chemistry process.

Following the 2^{nd} positive spacer wet chemistry treatment of step 360 is a step 370 of covalently immobilizing heparin to at least the blood contacting surface with a wet chemistry treatment including heparin. Preferably the Heparin covalent immobilization wet chemistry treatment is 0.1 to 2 mg/ml, and preferred 0.4 to 1.4 mg/ml, deaminated heparin, in Sodium chloride solution at pH 3.5-4.5. Additionally sodium cyanoborohydrite may be added in amounts of 0.01 to 0.1% w to the heparin solution for the wet chemistry around 5 to 15 minutes prior to circulation of the heparin solution wet chemistry treatment of step 370. The heparin wet chemistry treatment of step 370 is preferably at an elevated temperature of at least 50° C., preferably 60° C.-65° C., and is recirculated through the device(s) 40 for a circulation time of 60-180 minutes, preferably 90-150 minutes. The heparin wet chemistry treatment of step 370 then includes a non-recirculating 1^{st} rinse of about 3× priming volume of DI water at room temperature. Following the initial water rinse the heparin wet chemistry treatment of step 370 may include a sodium chloride solution (0.2-0.8M Sodium Chloride) rinse which is recirculated at ambient conditions for 5-20 minutes, and then this is followed by non-recirculating 2^{nd} water rinse of about 3× priming volume of DI water at room temperature. The heparin wet chemistry treatment of step 370 then may, if desired, include a quality control check of taking an in-line sacrificial tube section and stain the section and maintaining the results as a record of the wet chemistry process.

Following the heparin wet chemistry treatment of step 370 is a drying step 380 that may include a pressured gas flush of the devices 40 with an inert gas such as argon or nitrogen or air for 5 to 10 minutes, followed by clean filtered dry air a period of 8 to 24 hours to complete dehydration. The drying of step 380 then may, if desired, include a quality control check of taking an in-line sacrificial tube section and stain the section and maintaining the results as a record of the wet chemistry process, and this may be followed by testing or measuring of heparin bioactivity in the devices 50.

An alternative to the above process is not utilizing the negative spacer of step 350 and the 2^{nd} positive spacer of step 360. In this engineered heparin matrix only a single PEI spacer is present. The process may be described as the same as above but not including the cross linker in step 340 and thereafter proceeding directly to the heparin wet chemistry treatment of step 370 while skipping steps 350 and 360, an alternative explanation is that the process skips from the enhancing step 330 to the second positive spacer step 360 Skipping steps 340-350. Both descriptions of this alternative arrangement are accurate and simply define a single positive spacer.

The method of the present invention is universally applicable in that it defines a method of manufacturing medical products 50 with the ability to meet standard physiologically significant bioactivity with increased stability for clinical applications on a collection of substrates.

The preferred embodiments described above are illustrative of the present invention and not restrictive hereof. It will be obvious that various changes may be made to the present invention without departing from the spirit and scope of the invention. The precise scope of the present invention is defined by the appended claims and equivalents thereto.

What is claimed is:

1. A method of manufacturing a medical product having an engineered heparin bioactive matrix for clinical application on a blood contacting surface comprises the steps of:
a) activating a blood contacting surface of at least one component of a medical device via one of plasma treatment or gas activation;
b) assembling the medical product;
c) setting up the assembled medical device for wet chemistry in which wet chemistry treatments follows a blood flow path through device;
d) enhancing at least the blood contacting surface with a wet chemistry treatment including an aqueous solution having a strong oxidizing agent;
e) adding a positively charged spacer molecule to at least the blood contacting surface with a wet chemistry treatment including an aqueous solution having a cationic polymer; and
f) covalently immobilizing heparin to at least the blood contacting surface with a wet chemistry treatment including heparin.

2. The method of manufacturing a medical product having an engineered heparin bioactive matrix for clinical application according to claim 1 wherein the step of enhancing at least the blood contacting surface with a wet chemistry treatment including an aqueous solution having a strong oxidizing agent includes ammonium persulfate.

3. The method of manufacturing a medical product having an engineered heparin bioactive matrix for clinical application according to claim 2 wherein the step of adding a positively charged spacer molecule to at least the blood contacting surface with a wet chemistry treatment including an aqueous solution having a cationic polymer utilizes PEI as the spacer molecule.

4. The method of manufacturing a medical product having an engineered heparin bioactive matrix for clinical application according to claim 1 wherein the step of adding a positively charged spacer molecule to at least the blood contacting surface with a wet chemistry treatment including an aqueous solution having a cationic polymer with a cross linker, followed by adding a negatively charged spacer molecule to at least the blood contacting surface with a wet chemistry treatment including an aqueous solution having a anionic polymer, and followed by a step of adding a second positively charged spacer molecule to at least the blood contacting surface with a wet chemistry treatment including an aqueous solution having a cationic polymer.

5. The method of manufacturing a medical product having an engineered heparin bioactive matrix for clinical application according to claim 4 wherein at least one of the steps of adding a positively charged spacer molecule to at least the blood contacting surface with a wet chemistry treatment including an aqueous solution having a cationic polymer utilizes PEI as the spacer molecule.

6. The method of manufacturing a medical product having an engineered heparin bioactive matrix for clinical application according to claim 5 wherein by adding a negatively charged spacer molecule to at least the blood contacting surface with a wet chemistry treatment including an aqueous solution having an anionic polymer wherein dextran sulfate is utilized as the negatively charges spacer.

7. The method of manufacturing a medical product having an engineered heparin bioactive matrix for clinical application according to claim 4 the step of enhancing at least the blood contacting surface with a wet chemistry treatment including an aqueous solution having a strong oxidizing agent includes ammonium persulfate.

8. The method of manufacturing a medical product having an engineered heparin bioactive matrix for clinical application according to claim 1 further including quality control steps of staining sacrificial inline tubing sections before wet chemistry treatments and after at least one wet chemistry treatment.

9. The method of manufacturing a medical product having an engineered heparin bioactive matrix for clinical application according to claim 1 further including a drying step after the step of covalently immobilizing heparin to at least the blood contacting surface with a wet chemistry treatment including heparin, wherein the drying step includes a pressured gas flush of the medical product.

10. The method of manufacturing a medical product having an engineered heparin bioactive matrix for clinical application according to claim 1 wherein the step of activating a blood contacting surface of at least one component of a medical device includes a plasma treatment.

11. The method of manufacturing a medical product having an engineered heparin bioactive matrix for clinical application according to claim 10 wherein the plasma treatment step of activating a blood contacting surface of at least one component of a medical device utilizes propene, TMDSO or acrylic acid.

12. The method of manufacturing a medical product having an engineered heparin bioactive matrix for clinical application according to claim 1 wherein the step of activating a blood contacting surface of at least one component of a medical device includes an ozone gas treatment.

13. The method of manufacturing a medical product having an engineered heparin bioactive matrix for clinical application according to claim 1 wherein the medical device is one of a blood oxygenator, a dialysis catheter and a dialysis machine.

14. The method of manufacturing a medical product having an engineered heparin bioactive matrix for clinical application according to claim 13 wherein the medical device is a tubular blood oxygenator and wherein the step of assembling the medical product includes a winding station forming a tubular fiber bundle for the tubular blood oxygenator, a potting station for the tubular fiber bundle, a toming station for the potted fiber bundle and an assembly station for assembling the trimmed potted fiber bundle and other components of the tubular blood oxygenator.

15. The method of manufacturing a medical product having an engineered heparin bioactive matrix for clinical application according to claim 14 wherein the steps a) through f) occur in the same clean room.

16. The method of manufacturing a medical product having an engineered heparin bioactive matrix for clinical application according to claim 14 wherein the step of adding a positively charged spacer molecule to at least the blood contacting surface with a wet chemistry treatment including an aqueous solution having a cationic polymer with a cross linker, followed by adding a negatively charged spacer molecule to at least the blood contacting surface with a wet chemistry treatment including an aqueous solution having an anionic polymer, and followed by a step of adding a second positively charged spacer molecule to at least the blood contacting surface with a wet chemistry treatment including an aqueous solution having a cationic polymer.

17. The method of manufacturing a medical product having an engineered heparin bioactive matrix for clinical application according to claim 16 wherein at least one of the steps of adding a positively charged spacer molecule to at least the blood contacting surface with a wet chemistry treatment including an aqueous solution having a cationic polymer utilizes PEI as the spacer molecule.

18. The method of manufacturing a medical product having an engineered heparin bioactive matrix for clinical application according to claim 17 wherein by adding a negatively charged spacer molecule to at least the blood contacting surface with a wet chemistry treatment including an aqueous solution having a anionic polymer wherein dextran sulfate is utilized as the negatively charges spacer, and wherein the step of enhancing at least the blood contacting surface with a wet chemistry treatment including an aqueous solution having a strong oxidizing agent includes ammonium persulfate.

19. A product made according to the process of claim 1.

20. A medical product for clinical application having a blood contacting surface with an engineered heparin bioactive matrix comprising a positively charged spacer molecule coupled to at least the blood contacting surface and covalently immobilizing heparin coupled to the positively charged spacer molecule.
